(19) United States
(12) Patent Application Publication
(10) Pub. No.: US 2010/0228150 A1
(43) Pub. Date: Sep. 9, 2010

(54) NEURO GUIDEWIRE

(75) Inventors: John M. Zimmerman, Chanhassen, MN (US); Bradley D. Seibert, St. Louis Park, MN (US)

Correspondence Address: WHYTE HIRSCHBOECK DUDEK S.C.;INTELLECTUAL PROPERTY DEPARTMENT, 33 East Main Street, Suite 300, Madison, WI, 53703-4655, US

(73) Assignee: LAKE REGION MEDICAL, INC., Chaska, MN (US)

(21) Appl. No.: 12/398,713

(22) Filed: Mar. 5, 2009

(51) Int. Cl. A61M 25/01

(52) U.S. Cl. 600/585

(57) ABSTRACT

A guide wire having dual coil on core configuration. The guide wire has a core wire with distal and proximal ends or segments wherein two coils are tandomly disposed on its distal end.

The more proximally-disposed of the distal coils is multifilar having from 4 to 15 filars all wound in one direction. The multifilar coil comprises a super elastic material. The more distal of the distally-disposed coils comprises a radiopaque material, the core wire terminating with an atraumatic tip. A guide wire of this invention is particularly well suited to navigation of the neuroanatomy.

### CROSS-REFERENCE TO RELATED APPLICATIONS

None

### BACKGROUND

This invention relates to guide wire apparatuses and to methods for using same. More specifically, the present invention relates to guide wire apparatuses with improved torque and flexure characteristics.

Catheter guidewires (hereafter “guide wires”) have been used for many years to “lead” or “guide” catheters to target locations in animal and human anatomy. This is typically done via a body lumen, for example, such as traversing luminal spaces defined by the vasculature to the target location. The typical conventional guide wire is from about 135 centimeters to 195 centimeters (or more) in length. The guide wire generally comprises a generally solid core wire and a distal coil spring or coil, often made from a radiopaque material. The core wire is tapered on the distal end to increase its flexibility. The coil spring is typically soldered to the core wire at a point where the inside diameter of the coil spring matches the outside diameter of the core wire. Platinum is selected for the coil spring because it provides radiopacity for better fluoroscopic or other radiologic imaging during navigation of the guide wire in the body, and because it is biocompatible.

Navigation of a guide wire through the anatomy is usually achieved with the assistance of radiographic imaging. This is conventionally done by viewing the guide wire in the body lumen using X-ray fluoroscopy or other comparable methods. The guide wire optionally can be provided with a tip that is curved or bent to a desired angle so as to deviate laterally a short distance. By proximal rotation of the guide wire the curved distal tip can be made to deviate in a selected direction from an axis of the guide wire about which it rotates.

In use a guide wire is inserted into a catheter so that the guide wire can be advanced so that its distal end protrudes out the distal end of the catheter, and also is pulled back in a proximal direction so as to be retracted into the catheter. Visualization is by fluoroscope, for example, or another device. The guide wire and catheter are introduced into a luminal space, comprising for example, a vessel or duct and advanced therethrough until the guide wire tip reaches a desired luminal branch. The user then twists the proximal end of the guide wire so as to rotate and point the curved distal tip into the desired branch so that the device may be advanced further into the anatomy via the luminal branch. The catheter is advanced over the guide wire to follow, or track, the wire. This procedure is repeated as needed to guide the wire and overlying catheter to the desired target location of medical interest.

A guide wire having a relatively low resistance to flexure yet relatively high torsional strength is very desirable. As the guide wire is advanced into the anatomy, internal resistance from the typically tortuous turns, and surface contact, decreases the ability to advance the guide wire further within the luminal space. This, in turn, may lead to a more difficult and prolonged procedure. A guide wire with high flexibility helps overcome the problems created by internal resistance. However, if the guide wire does not also have good torque characteristics (torsional stiffness), the user will not be able to twist the proximal end in order to rotate the distal tip of the guide wire as required. Flexibility and good torque transmission characteristics are especially needed when the guide wire is intended to be used in the highly complex neurovasculature and neuroanatomy.

This invention relates to medical guide wires which provide the desired performance characteristics. Yet more specifically, this invention relates to guide wires having distal and proximal coil on core construction wherein the distal coil comprises a radiopaque material and the proximal coil is a multifilar guide wire and comprises a super elastic material. A core wire or mandrel of this invention generally is not super elastic and preferably has a flattened extreme distal end coupled to an atraumatic tip.

### BRIEF DESCRIPTION OF THE FIGURES

The present invention will now be illustrated in the attached FIGS in which like numerals are used to designate like features, and in which:

FIG. 1 is a side view of a guide wire of the invention;

FIG. 2 is a sectional view of the guide wire of FIG. 1 taken along line 2-2 of FIG. 1;

FIG. 3 is a second sectional view of the guide wire of FIG. 1 rotated 90° from the view seen in FIG. 2;

FIG. 4 is an exemplary seven filar version of coil 14 in FIGS. 1-3;

FIGS. 5-7 illustrate a 12 filar version of coil 14 of FIGS. 1-3;

FIGS. 8-10 illustrate a 4 filar version of coil 14 of FIGS. 1-3;

FIG. 11 is an exploded version of the guide wire of FIGS. 1-3.

The above FIGS should be understood as illustrative and not limiting of the invention.

### DETAILED DESCRIPTION OF THE FIGURES

Thus, in FIG. 1 there is shown a guide wire 10, particularly a neurological guide wire, of this invention. The guide wire 10 in FIG. 1 is about the distal 50 cm of the wire, the remaining proximal core wire length 12 being substantially conventional in structure.

Shown in FIG. 1 are distal coil structures 14, 16. The details of extreme distal coil 16 and immediately proximal (to distal coil 16) coil 14 are discussed below. Core wire or mandrel 18 extends through tandomly-disposed distal coils 14, 16 and terminates at atraumatic rounded tip 20.

Generally speaking, core wire 18 comprises a non-super elastic material such a stainless steel or MP35N. Other core wire metals and alloys, optionally including hydrophilic, lubricious or hydrophobic coatings, may be used on proximal core wire length 12. Proximal core wire length 12 may include exchange wire coupler structures (not shown), see e.g., U.S. Pat. No. 5,282,478 to Fleischhacker, Jr., et al. and U.S. Pat. No. 5,546,958 to Thorud et al. Alternatively proximal core wire length 12 may comprise the rest of an exchange wire or other proximal guide wire structure known to the art (also not shown), potentially increasing guide wire length to 250 cm or more.

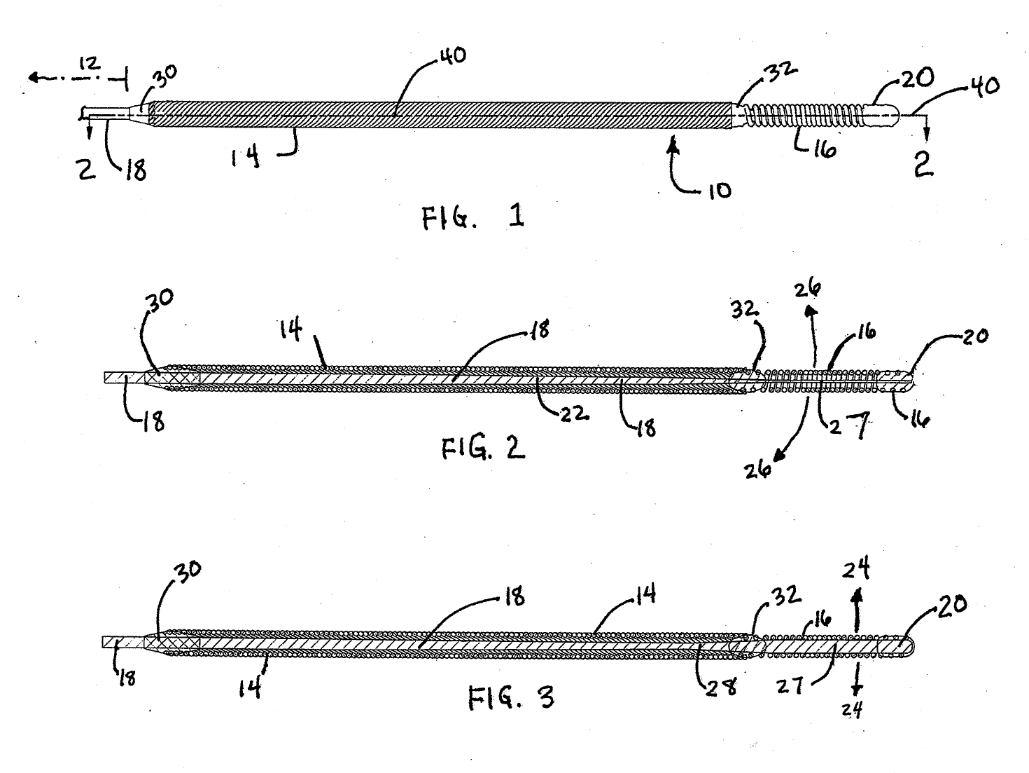

FIG. 2 shows in section the guide wire of FIG. 1 taken along line 2-2 of FIG. 1. Line 2-2 of FIG. 1 is approximately the central axis 40 of guide wire 10. Core wire 18 is shown to taper gradually left to right (right being the distal direction) starting at about 22. Core wire 18 then is optionally flattened 27 proximal to and is coupled to atraumatic tip 20 starting at 28 and extending to tip 20. (This is better shown in FIG. 3).

Optional flattening 27 of core wire 18 immediately proximate tip 20 provides an enhanced tip stiffness in a direction in the plane of the flattened tip and an enhanced floppiness in a direction perpendicular to the plane of the flattened core wire tip (arrows 24 and 26 respectively). Thus by rotation of the guide wire the medical professional can determine for herself whether the floppiness or stiffness feature of this invention (or both) are to be utilized. Flattening of core wire 27 is an optimal but preferred feature of a guide wire (especially a neuro guide wire) of this invention.

Core wire 18 is attached to coil structures 14, 16 by means of, for example, a solder joint 30, or an adhesive joint 32. Joints 30, 32 provide a smooth, gradual transition from coil wire 18 to coil 14 for interventional or diagnostic devices (e.g., a catheter) being guided or steered to a vascular site of medical interest. This is especially true of joint 30 where it is necessary to avoid an abrupt change in overall guide wire diameter so that a device passing thereover could encounter resistance. Joint 32, in addition to providing a smooth transition between coils 14, 16 (for any cooperating device) must also be flexible e.g., to bend with coil 18, so as not to produce a kink point at that juncture. Atraumatic tip 20 is usually a solder joint or bulb (it is bullet-shaped in this embodiment) and must also be smooth and rounded to prevent tissue injury as the tip 20 passes through the vasculature. Tip 20 may be made of other materials, adhesives, polymers, metals or alloys and may have other exterior shapes (e.g., spherical, bulbous) as long as such shapes are atraumatic to vascular structures. The distal end of coil 16 is coupled to core wire 18 with the same solder joint or bulb which creates and defines tip 20.

FIG. 3 shows a sectional view of FIG. 2 rotated 90° from that shown in FIG. 2. FIG. 3 shows core wire flattened distal tip 27 and the tapered section 22 of core wire 18 which couples to the lengthy conventional diameter of proximal core wire 12.

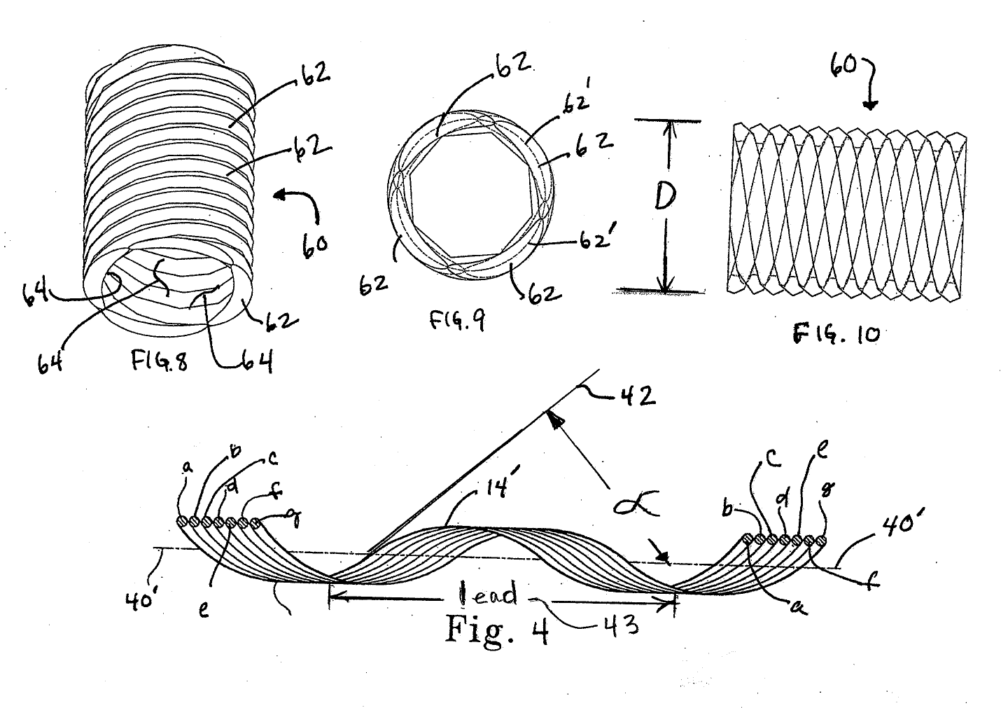

Coils 14, 16 and especially coil 14 constitute a particularly important feature of this invention. Coil 14 is multifilar, having between 4 and 15, preferably 5 to about 12 filars or wires helically wound in the same direction. A sectional view of a 7 filar embodiment of coil 14′ is shown in FIG. 4. The individual filars (letters a-g in FIG.4) are wound to be helically parallel and in close proximate contact with each other. The axis of coil 14′ is shown at 40′. The axis 40′ of coil 14′ is generally coincidental with, or at least parallel to, the central axis 40 of the guide wire 10 shown in FIG. 1. If wound to be in actual contact with each other, filars a through g provide a surprising and unexpected kink and camber resistance for the approximately 35 cm of the helically wound coil 14. That kink and camber resistance dramatically improves torque central of the distal portion of the guide wire. That improved torque control is especially significant when the present invention is used to navigate the often tortuous and complex neurovasculature or neuroanatomy.

There is shown in FIGS. 5 through 10, four and twelve filar versions of coil 14 of guide wire 10. FIG. 5 shows a perspective view of the 12 filar version 50 of coil 14, an end-on view being shown in FIG. 6. The helices 52 shown in phantom in FIG. 6 are down-stream from the solid line end view and would extend into the plane of the page. The 12 filars of coil 50 subtend each approximately 30° of the substantially circular open end 54 of coil 50 (defined by the twelve filars 56) indicating the filars are tightly wound and, as shown, adjacent helices are in substantial contact with each other. This tightly wound coil design is preferred with spaced helices being a less preferred approach. FIG. 7 is a side view of a segment of coil 14.

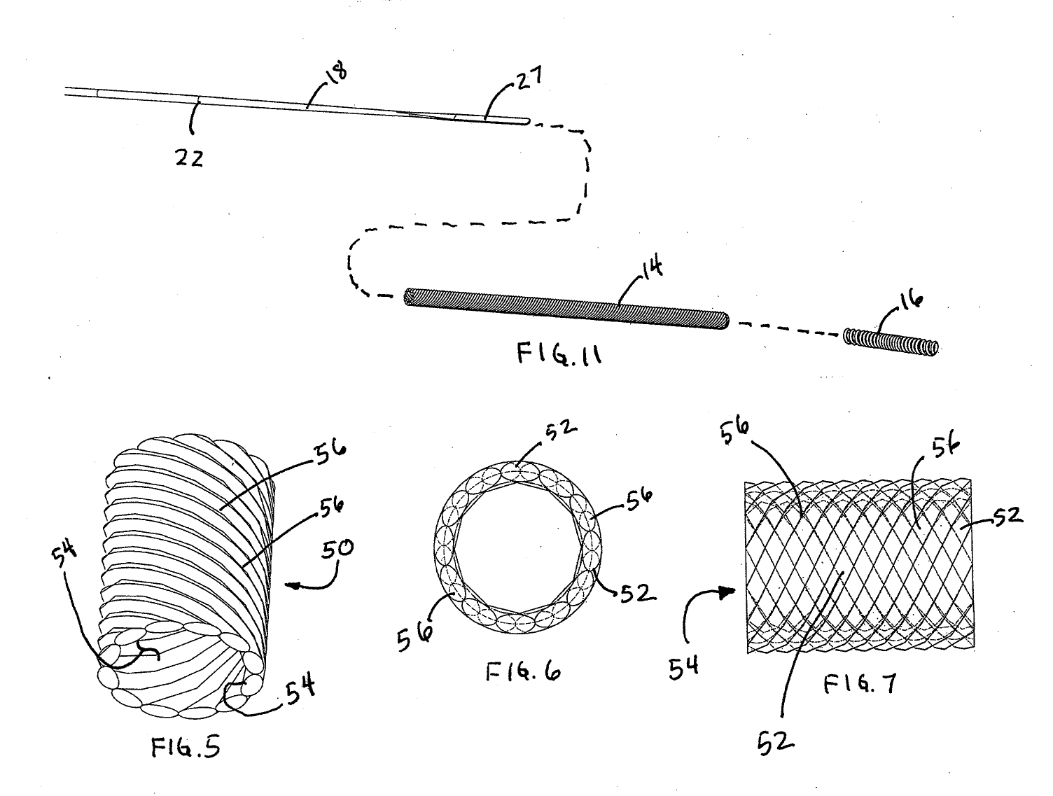

FIGS. 8, 9 and 10 show a four filar embodiment 60 of coil 14, the filars being designated 62. FIG. 8 is a perspective end view while FIG. 9 is an end-on view with “down-stream” helices 62′ being shown in phantom. Again the 4 filars shown in FIGS. 8, 9 and 10 are tightly wound, each helical filar being in substantial contact with both of its helically-adjacent neighbors to provide a preferred tightly wound coil. Each of the filars 62 of the 4 filar version of coil 60 of FIG. 8, as shown, provide about 90 degrees of the circular opening 64 of coil 60. FIG. 10 is a side view of a segment of a 4 filar coil 14.

FIG. 11 is an exploded view of core wire 18 and distal coils 14, 16. Core wire taper 22 and distal flattening 27 are shown. The components of FIG. 11 are soldered, or adhesively coupled as described from FIG. 1. An atraumatic tip is created by soldering distal coil 16 at its extreme distal end to the flattened segment 27 of core wire 18. This sectional structure is best seen in FIG. 2.

Of particular importance to obtaining the advantageous characteristics of the present invention is the helical angle defined by the substantially parallel lines of contact between adjacent helices of the coil. The helical angle is defined as the angle between a line of closest approach between adjacent or neighboring helices of the coil and a plane which includes and could rotate around the central axis of the coil (shown at 40, 40′ in FIGS. 1 and 4).

The determination of helical angle or alpha (α) angle is shown in FIGS. 4 and 10. In FIG. 4, (α) angle is the angle between coil axis 40′-40′ and a line 42 which is parallel to one edge of one of the filars “a.” Also shown in FIG. 4 is lead length or lead 43 i.e., the linear length of a 360° cycle or portion of coil 14′. In FIG. 10 coil diameter “D” is shown. Alpha (α) angle then is calculated as follows:

$$Alpha angle = arc tan ( \frac{lead}{Π \times D} )$$

Illustrating the above calculation, assuming a 0.0015 inch filar diameter, dimension “D” of 0.014′, and a tightly wound coil the calculated α angles would be as follows:

| | |
|---|---|
| 4 filar, | 7.8° |
| 7 filar | 13.4° |
| 12 filar | 22.3° |
| 15 filar | 27.1° |

Alpha helical angle is determined by the diameter of the filars, the tightness of the wrap and the number of filars in the coil. Generally speaking the helical angle of coils 14 of this invention will fall in the range of 5° to about 35°, preferably 6° to about 30°, and most preferably 7° to about 25°.

Multifilar coil 14 which is immediately proximal to coil 16 but is still disposed on the distal portion or segment of core wire 18 and comprises a superelastic material. Super elastic materials as the term is used herein are well known to this art. The preferred superelastic material for use herein is a nickel titanium alloy commonly known as nitinol. While metallic superelastic materials are well known, non-metallic e.g., polymeric, materials having the performance characteristics of super elastic metallic alloys could be used.

The vasculature that feeds the neuro-anatomy is referred to as the neuro-vasculature. The neuro-vasculature is deemed to begin at either the aortic branch (left carotid artery) or the brachiocephalic artery (right carotid artery). Near the jaw the carotid bifurcates into the external and internal carotid artery. The external carotid feeds the outer portions of the face and temporal regions such as the jaw and face. The internal carotid feeds the inner portion of the brain. It provides the life sustaining blood flow to the functioning part of the brain. Both branches are considered tortuous and difficult to navigate through with a guide wire.

The internal carotid artery has been mapped out to include common curvatures that are referred to as the Cervical segment (C1), Petrous segment (C2), Lacerum segment (C3), Cavernous segment (C4), Clinoid segment (C5), Ophthalmic segment (C6) and finally the Communicating segment (C7). At this point the vasculature joins into a feature called the Circle of Willis. These are a series of arteries that are anywhere from 1-4 mm in diameter and can have curvatures ranging in 3-7 mm in radius. These turns and other complicated arterial and venous structures of the neuroanatomy are difficult to navigate with a guide wire and commonly cause guide wires to camber and possibly kink. This creates complications when trying to steer the guide wire to the diseases location. The present guide wire has been found to be particularly advantageously used in navigating the neuro-vasculature.

We claim:

1. A guide wire with coil on core construction, the guidewire comprising:
an elongate core wire having distal and proximal ends, the core wire having:
two helically-wound coils tandomly disposed around the core wire distal end and being in substantially abutting relationship, the more distally-disposed distal coil comprising a radiopaque material, the more proximally disposed distal coil comprising a super elastic material, the super elastic coil being of a multifilar construction having between 4 and 15 filars all filars being wound in the same direction, the distal end of the core wire terminating in;
an atraumatic tip.

2. A guide wire according to claim 1 wherein the core wire comprises a non-super elastic material.

3. A guide wire according to claim 1 wherein the core wire comprises stainless steel.

4. A guide wire according to claim 1 wherein the core wire consists essentially of stainless steel.

5. A guide wire according to claim 1 the more distally-disposed tandem distal coil comprises metal selected from the group consisting of platinum, palladium, or tantalum.

6. A guide wire according to claim 1 wherein the more proximally-disposed distal coil comprises a super elastic material.

7. A guide wire according to claim 1 wherein the more proximally-disposed distal coil consists essentially of a super elastic material.

8. A guide wire according to claim 1 wherein the more proximally-disposed distal coil comprises a nickel titanium alloy.

9. A guide wire according to claim 1 wherein the more proximally-disposed distal coil comprises a super elastic material and has between 5 and 12 filars.

10. A guide wire according to claim 1 wherein the more proximally-disposed distal coil comprises 12 filars.

11. A guide wire according to claim 1 wherein the more proximally-disposed distal coil comprises a nickel-titanium alloy super elastic material.

12. A guide wire according to claim 1 wherein the more proximally-disposed distal coil comprises nitinol.

13. A guide wire according to claim 1 wherein the core wire is a monofilament.

14. A guide wire according to claim 1 wherein the more proximally-disposed distal coil comprises a uni-directionally wound multifilar coil wherein individual coils define a helix angle falling in the range of about 5° to about 35°.

15. A guide wire according to claim 1 wherein the more proximally-disposed distal coil comprises a uni-directionally wound multifilar coil wherein individual coils define a helix angle falling in the range of about 6° to about 30°.

16. A guide wire according to claim 1 wherein the more proximally-disposed distal coil comprises a uni-directionally wound multifilar coil wherein individual coils define a helix angle falling in the range of about 7° to about 25.°

17. A guide wire according to claim 1 wherein the more proximally-disposed distal coil comprises a uni-directionally wound multifilar coil, the individual filars of the coil being wound so that each filar is in contact with its neighboring filars.

18. A guide wire according to claim 1 wherein the core wire has a substantially flattened segment immediately proximal to the atraumatic tip.

19. A guide wire according to claim 1 wherein the core wire has one or more reductions in diameters in its distal end.

20. A guide wire according to claim 1 wherein the core wire has a proximal end diameter, has a reduction in diameter in its distal end, a flattened segment coupled to the reduced diameter distal end which is coupled to the atraumatic tip.

21. A guide wire according to claim 1 wherein the guide wire is a neurological guide wire.
